(19) United States
(12) Patent Application Publication
(10) Pub. No.: US 2019/0200859 A1
(43) Pub. Date: Jul. 4, 2019

(54) PATTERNED BEAM ANALYSIS OF IRIDOCORNEAL ANGLE

(71) Applicant: BROADSPOT IMAGING CORP, Richmond, CA (US)

(72) Inventor: Tushar M. RANCHOD, Berkeley, CA (US)

(73) Assignee: BROADSPOT IMAGING CORP, Richmond, CA (US)

(21) Appl. No.: 16/231,047

(22) Filed: Dec. 21, 2018

Related U.S. Application Data
(60) Provisional application No. 62/611,066, filed on Dec. 28, 2017.

(51) Int. Cl. A61B 3/12; H04N 5/247; H04N 5/225; H04N 5/232; A61B 3/14; A61B 3/00

(52) U.S. Cl. CPC A61B 3/1216; H04N 5/247; A61B 3/0008; H04N 5/23238; A61B 3/14; H04N 5/2256

(57) ABSTRACT

An optical imaging device may include a support structure and a plurality of imaging channels, where each of the imaging channels includes a discrete optical imaging pathway disposed within the support structure. Additionally, the imaging channels may be aimed at different angles relative to each other. Further, illumination sources may correspond respectively to the imaging channels, where each illumination source emits an illumination pattern along a discrete optical illumination pathway positioned non-coaxially relative to the discrete optical imaging pathway of each imaging channel. The optical imaging device also includes image capturing devices, where each image capturing device is respectively associated with one of the imaging channels to capture digital photograph images of respective portions of an iridocorneal angle with topographical information revealed by the illumination sources.

### FIELD

The application relates generally to patterned beam analysis of the iridocorneal angle.

### BACKGROUND

Ocular imaging is commonly used both to screen for diseases and to document findings discovered during clinical examination of the eye. Imaging of the anterior segment of the human eye may be used to document pathology of the anterior segment, including the iridocorneal angle of the eye. Documentation and analysis of the iridocorneal angle may be relevant to a myriad of various types of patients, including patients diagnosed with glaucoma, patients who are labeled as glaucoma suspects, patients who have undergone and may undergo glaucoma surgical procedures, patients with proliferative ischemic retinal diseases, patients with tumors of the anterior segment, and patients with blunt traumatic injury to the eye. The iridocorneal angle may be obscured from direct view on clinical examination by internal reflection of the cornea.

The subject matter claimed herein is not limited to embodiments that solve any disadvantages or that operate only in environments such as those described above. Rather, this background is only provided to illustrate one example technology area where some embodiments described herein may be practiced.

### SUMMARY

Embodiments of the present disclosure discuss an optical imaging device. The optical imaging device may include a support structure and a group of imaging channels. In some embodiments, each imaging channel of the group of imaging channels may include a discrete optical imaging pathway, and the group of imaging channels may be disposed within the support structure. Additionally, in some embodiments, the group of imaging channels may be aimed at different angles relative to each other. The optical imaging device may also include a group of illumination sources corresponding respectively to the group of imaging channels. In some embodiments, each illumination source of the group of illumination sources may be configured to emit an illumination pattern along a discrete optical illumination pathway positioned non-coaxially relative to the discrete optical imaging pathway of each imaging channel of the group of imaging channels. Further, the optical imaging device may include a group of image capturing devices. In some embodiments, each image capturing device of the group of image capturing devices may be respectively associated with one of the group of imaging channels to capture digital photograph images of respective portions of an iridocorneal angle of an eye to generate a topographical profile of the iridocorneal angle revealed by the group of illumination sources.

The objects and advantages of the embodiments will be realized and achieved at least by the elements, features, and combinations particularly pointed out in the claims.

Both the foregoing general description and the following detailed description are given as examples and are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1A illustrates a cross-sectional front view of an optical imaging device for imaging an eye;

FIG. 1B illustrates a cross-sectional side view of an eye, including portions of the optical imaging device in FIG. 1A for imaging an iridocorneal angle of the eye;

FIG. 2A illustrates a front schematic view of the eye of FIG. 1B, including an embodiment with multiple example optical pathways for imaging the iridocorneal angle of the eye;

FIG. 2B illustrates a front schematic view of the eye of FIG. 1B, including another embodiment with multiple example optical pathways for imaging the iridocorneal angle of the eye;

FIG. 2C illustrates a front schematic view of the eye of FIG. 1B, including yet another embodiment with multiple example optical pathways for imaging the iridocorneal angle of the eye;

FIG. 2D illustrates a front schematic view of the eye of FIG. 1B, including yet another embodiment with multiple example optical pathways for imaging the iridocorneal angle of the eye;

FIG. 2E illustrates a front schematic view of the eye of FIG. 1B, including yet another embodiment with multiple example optical pathways for imaging the iridocorneal angle of the eye;

FIG. 3A illustrates example image data of a portion of an iridocorneal angle obtained using diffuse illumination;

FIG. 3B illustrates example image data of the portion of the iridocorneal angle in FIG. 3A obtained using patterned illumination;

FIG. 3C illustrates example image data of the portion of the iridocorneal angle in FIG. 3A obtained using multiple-patterned illumination;

FIG. 3D illustrates example image data of the portion of the iridocorneal angle in FIG. 3A obtained using differing patterns in multiple-patterned illumination; and

FIG. 4 illustrates an example system that may be used in patterned beam analysis of the eye.

### DESCRIPTION OF EMBODIMENTS

Patterned beam analysis of the iridocorneal angle may indicate a cross-sectional profile or a topographical profile of eye tissue that is part of the iridocorneal angle. For example, the iridocorneal angle may include an angle formed between the iris and the cornea at a periphery of the anterior chamber of the eye. Many types of patients such as patients diagnosed with, or patients whom are at risk of, glaucoma or diabetes in addition to eye-trauma patients may benefit from analysis and documentation of the iridocorneal angle, including the cross-sectional profile and/or the topographical profile of the iridocorneal angle.

Viewing and imaging of the iridocorneal angle, at least directly, may be obscured due to anatomy of the eye and internal reflection of the cornea. Some practices for examining the iridocorneal angle may include using a contact lens with multiple mirrors or prisms where the mirrors or prisms may be positioned to help avoid total internal reflection while helping to provide views of the iridocorneal angle. In other applications, ophthalmologists may sometimes use a Koeppe direct gonioscopic lens, which may help to allow for visualization of the iridocorneal angle without the assistance of mirrors or prisms. Such practices incorporating a gonioscopic optical system may provide a direct (e.g., en face) view of the iridocorneal angle. As used herein, the term “en face” may refer to a view from approximately the center of the pupil and directed towards the iridocorneal angle.

En face imaging may identify abnormal vasculature as well as pigmentary patterns, which are visible in gonioscopic photography with various landmarks of the iridocorneal angle. The en face view with pigmentation may not be replicated by anterior segment optical coherence tomography (OCT), but such an en face view from gonioscopy also does not provide accurate quantitative evaluation of the angle structure or topographic profile. By comparison, some technologies such as optical coherence tomography (OCT) may provide topographical (e.g., cross-sectional or three-dimensional images) of the iridocorneal angle.

Both en face and topographical information about the iridocorneal angle may be clinically valuable. For example, topographical evaluation may identify specific contours of the iridocorneal angle and iris, as well as distances or angles between anterior segment structures. However, OCT technology is not digital photography, and OCT may incorporate methods using orthogonal illumination and interferometry to topographically profile the iridocorneal angle, methods which may lead to increased costs for manufacturers, clinicians, and patients, and which in turn, may lead to decreased access to topographical information about the iridocorneal angle. Furthermore, as noted above, such a procedure does not yield an en face view of the iridocorneal angle.

Some embodiments described in this disclosure may include an optical imaging device for digitally imaging the topographical profile of the iridocorneal angle. For example, in some embodiments, the optical imaging device may include imaging channels housing one or more components for imaging the eye, including the iridocorneal angle. The imaging channels may include one or more image capturing devices (e.g., camera sensors), optical lenses, one or more optical prisms, one or more illumination sources, and other suitable components for optical imaging. In some embodiments, the illumination source may emit an illumination pattern used to illuminate a section of the iridocorneal angle. Emission of the illumination pattern may occur along an optical illumination pathway that is non-coaxial with an optical imaging pathway. In this manner, the illumination pattern may reveal or highlight topographical features of that section of the iridocorneal angle that may, in turn, be captured by at least one of the image capturing devices. For example, by illuminating along an optical illumination pathway and imaging along an optical imaging pathway, shadows cast by the illumination along the optical illumination pathway may be observed due to variations in the topographical features of the eye, such as the iris and/or the iridocorneal angle. Additionally or alternatively, using a known variation between the optical illumination pathway and the optical imaging pathway and by automatically measuring the observed shadows, a topographical profile of the iridocorneal angle may be generated. Examples of various embodiments are described in greater detail below. For example, when imaging a portion of the eye, optical illumination may occur: along one or more optical illumination pathways positioned within one imaging channel or multiple imaging channels that are angled relative to each other; along multiple optical illumination paths simultaneously, sequentially, or at other different times; and/or in any suitable combination thereof.

In some embodiments, the optical imaging device may include multiple illumination sources, for example, at least one illumination source corresponding to each of multiple optical imaging channels. Thus, in some embodiments, the multiple illumination sources may be used to reveal a topographic profile of up to a three hundred and sixty degree view of the iridocorneal angle. For example, the multiple illumination sources may each emit the illumination pattern simultaneously or in rapid succession, thereby allowing the corresponding optical imaging channels to capture the revealed topographic profile of their respective sections. Additionally or alternatively, the optical imaging channels may be positioned in an overlapping manner such that captured images of various sections of the iridocorneal angle may be stitched together to form a composite image, including the cross-sectional profile of up to three hundred and sixty degrees of the iridocorneal angle.

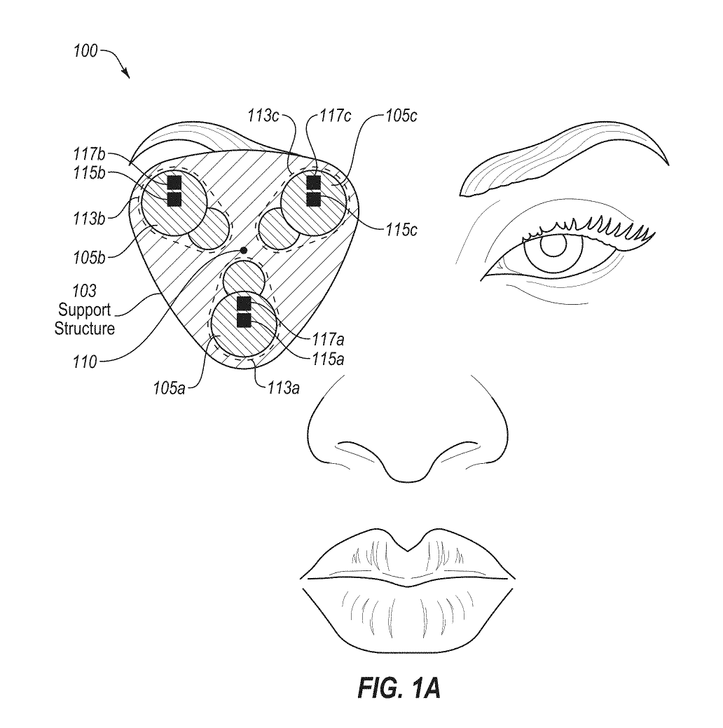

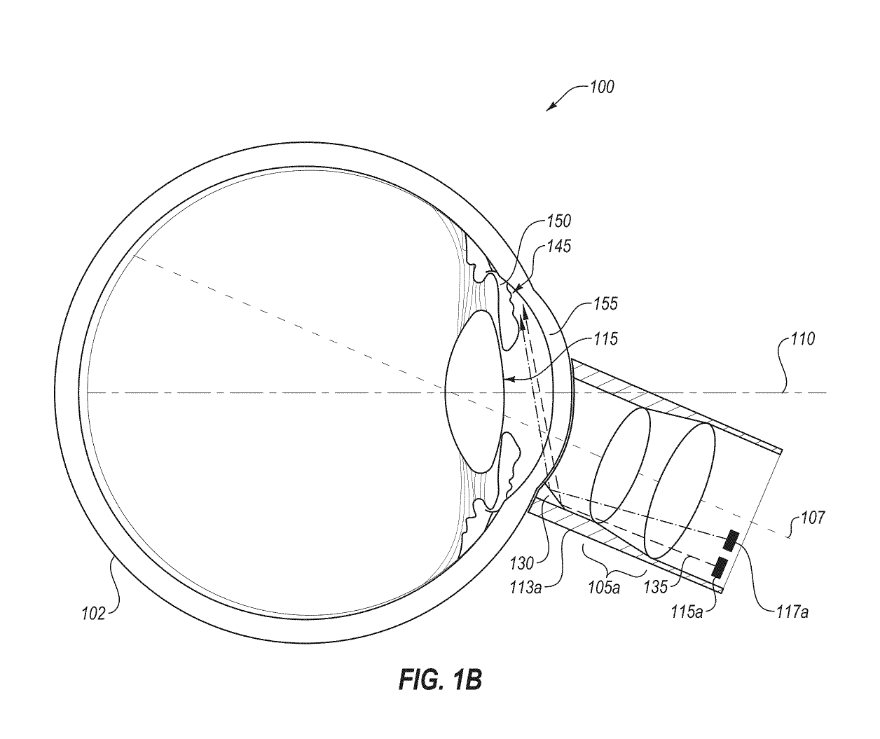

FIG. 1A illustrates a cross-sectional front view of an optical imaging device 100 for imaging an eye, where the optical imaging device 100 is arranged according to one or more embodiments of the present disclosure. As illustrated, the optical imaging device 100 includes a support structure 103, lenses 105a-105c, imaging channels 113a-113c, image capturing devices 115a-115c, and/or illumination sources 117a-117c. In some embodiments, the optical imaging device 100 may be aligned relative to a central axis 110 of an eye.

The support structure 103 may be the same as or similar to the support structure described in U.S. patent application Ser. No. 16/217,750 entitled MULTIPLE OFF-AXIS CHANNEL OPTICAL IMAGING DEVICE UTILIZING UPSIDE-DOWN PYRAMIDAL CONFIGURATION filed on Dec. 12, 2018, the contents of which are hereby incorporated by reference in their entirety. In these or other embodiments, the support structure 103 may house the lenses 105a-105c, the imaging channels 113a-113c, the image capturing devices 115a-115c, and the illumination sources 117a-117c. Additionally or alternatively, the support structure 103 may be sized and/or shaped for ergonomic purposes, e.g., to more suitably interface with facial features of a patient.

In some embodiments, the lenses 105a-105c may focus, disperse, and/or otherwise alter light transmission to enhance imaging capability of the image capturing devices 115a-115c. More or fewer numbers of lenses 105 may be used within any of the imaging channels 113, e.g., to permit more suitable imaging of a particular area of the eye. Additionally or alternatively, the lenses 105 may be sized and shaped to fill an inner diameter of the imaging channels 113 that house the lenses 105, while in other embodiments, the lenses 105 may be sized and shaped to be less than the inner diameter of the imaging channel 113. Additionally or alternatively, one or more components may be positioned between, adjacent to, distal to, and/or proximal to any of the lenses 105.

In some embodiments, the imaging channels 113a-113c may be angled relative to each other. Additionally or alternatively, the imaging channels 113a-113c may be angled relative to the central axis 110 of the eye such that no imaging channel 113 may be coaxial with the central axis 110 of the eye. However, in some embodiments, at least one imaging channel 113 may be coaxial with the central axis 110 of the eye. The imaging channels 113a-113c may be sized, shaped and/or positioned within the support structure 103 in any suitable configuration, e.g., depending on an imaging application or pupil size of the eye to be imaged. Additionally or alternatively, the imaging channels 113a-113c may be sized, shaped and/or positioned relative to the eye, e.g., the central axis 110 of the eye depending on an imaging application or pupil size of the eye to be imaged. For example, in some embodiments, other areas of the eye besides the iridocorneal angle may be imaged, such as the cornea, the iris, the sclera, the retina, and any other suitable area of the eye, whether in the anterior or posterior chamber of the eye.

Additionally or alternatively, more or fewer imaging channels 113 may be utilized in the optical imaging device 100, e.g., to facilitate up to three hundred and sixty degrees around the eye of image acquisition capability. For example, the optical imaging device 100 may include imaging channels 113 numbering between two and twelve imaging channels 113, such as between two and three, three and four, four and five, five and six, six and seven, seven and eight, eight and nine, or nine and ten. In some embodiments, more imaging channels 113 may be utilized to provide a more circumferential view of the iridocorneal angle while less imaging channels 113 may provide less of a circumferential view of the iridocorneal angle, given that each imaging channel 113 may only capture a portion of the iridocorneal angle. In these or other embodiments, the image capturing devices 115 may capture images all at the same time or in rapid succession, for example, using a rapid multi-plex. In this manner, for example, topographical information or a topographical profile may be generated at representative locations, e.g., at 12 o'clock, 2 o'clock, 4 o'clock, 6 o'clock, 8 o'clock, and 10 o'clock positions of the eye. Additionally or alternatively, one or more of the imaging channels 113 may be rotated relative to the support structure 103. For example, while the support structure 103 remains in a static position relative to the eye and/or facial features of the patient, any of the imaging channels 113 may be rotated inside the support structure 103. Such internal rotation of the imaging channels 113 may enable different portions and/or perspectives of the eye to be imaged.

In some embodiments, the image capturing devices 115a-115c may include camera sensors such as an entire imaging sensor or a portion of a larger digital camera, where the larger digital camera may be positioned outside of the optical imaging device 100. Additionally or alternatively, more or fewer image capturing devices 115 may be utilized in the optical imaging device 100, e.g., depending on an imaging application or pupil size of the eye to be imaged.

In some embodiments, the illumination sources 117a-117c may include any light emitting device configured to transmit an optical signal along a corresponding optical illumination pathway. In these or other embodiments, the illumination sources 117a-117c may emit positive illumination (e.g., radiated or reflected light) and/or negative illumination (e.g., at least partially occluded or blocked light). Additionally or alternatively, the illumination sources 117a-117c may emit patterned illumination, for example, in the form of a slit, an “X”, an asterisk (*), a star, a plus (+) symbol, a minus (−) symbol, a “T”, a polka dot pattern, and/or any other suitable pattern.

Modifications, additions, or omissions may be made to the embodiments of FIG. 1A without departing from the scope of the present disclosure. For example, in some embodiments, the support structure 103 may include any number of other components that may not be explicitly illustrated or described. Additionally or alternatively, the support structure 103 may be sized, shaped, and/or oriented relative to facial features in other suitable ways than may be explicitly illustrated or described. Additionally or alternatively, for example, the imaging channels 113a-113c may be sized, shaped, positioned, and/or oriented within the support structure 103 in other suitable ways than may be explicitly illustrated or described.

FIG. 1B illustrates a cross-sectional side view of an eye 102, including portions of the optical imaging device 100 in FIG. 1A for imaging an iridocorneal angle 145 of the eye 102, all arranged according to one or more embodiments of the present disclosure. As illustrated in FIG. 1B, the imaging device 100 includes the lenses 105a, the imaging channel 113a, the image capturing device 115a, and the illumination source 117a of FIG. 1A, in addition to a prism 130, an optical imaging pathway 135, an optical illumination pathway 140, and/or a center channel axis 107a. FIG. 1B also illustrates example features of the eye 102, including the central axis 110, an iridocorneal angle 145, an iris 150, and a cornea 155.

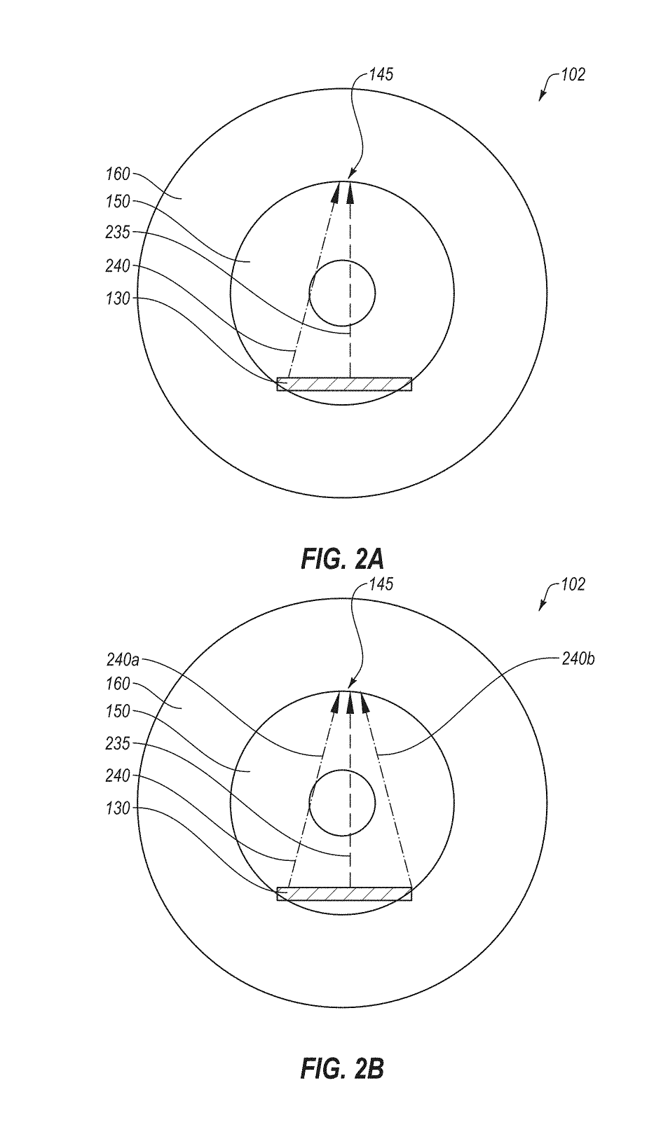

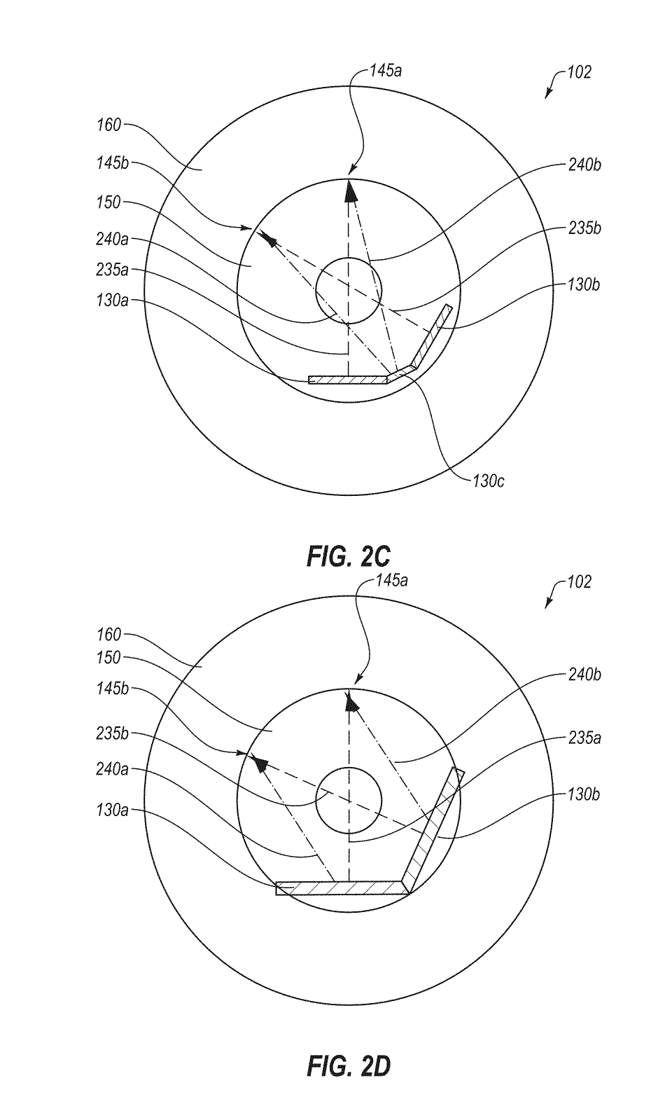

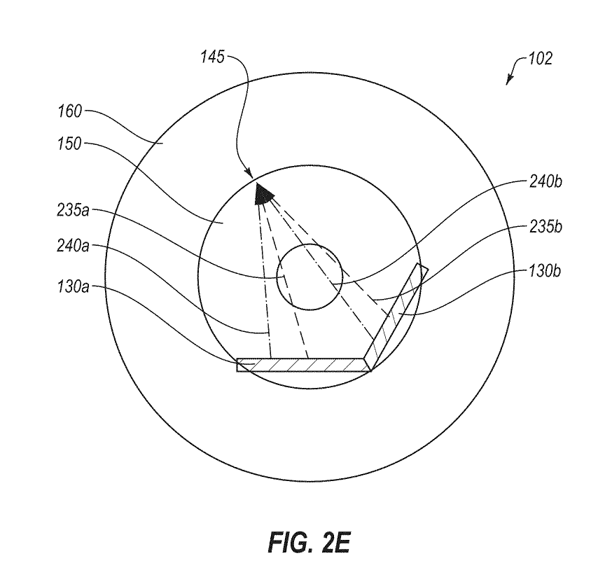

In some embodiments, the prism 130 may be configured as a mirror, beam splitter, or other suitable reflective element (e.g., partially reflective, substantially reflective, or completely reflective). In these or other embodiments, multiple prisms 130 may be positioned within an imaging channel 113, while in other embodiments, only a single prism 130 within an imaging channel 113. In some embodiments, the prism 130 may help direct light to and/or from the eye 102, e.g., permitting multi-directional travel of optical signals between the eye 102 and the optical imaging device 100. For example, the prism 130 may at least partially direct one or both of the optical imaging pathway 135 and the optical illumination pathway 140 toward the iridocorneal angle 145 of the eye 102. Accordingly, in some embodiments, the optical imaging pathway 135 may proceed from the image capturing device 115a, to the prism 130, and then to the iridocorneal angle 145. Additionally or alternatively, in some embodiments, the optical illumination pathway 140 may proceed from the illumination source 117a, to the prism 130, and then to the iridocorneal angle 145 (e.g., through an anterior chamber of the eye 102). In these or other embodiments, from the iridocorneal angle 145, light may be reflected back to one or more components of the optical imaging device 100. For example, from the iridocorneal angle 145, light may be reflected back to the prism 130 and the image capturing device 115a. In some embodiments, in the event of multiple optical imaging pathways (e.g., the optical imaging pathways 235 of FIGS. 2B-2E), the multiple optical imaging pathways may be configured to share the image capturing device 115a as a common image sensor.

In some embodiments, the optical imaging pathway 135 may include a path along which the image capturing device 115a is configured to obtain image data. Additionally or alternatively, the optical imaging pathway 135 may include an optical path that the image capturing device 115a utilizes to image target portions of the eye 102, such as the iridocorneal angle 145. In these or other embodiments, the image capturing device 115a may be positioned anywhere within the imaging channel 113a and directed at any angle relative to the center channel axis 107a. Additionally or alternatively to the image capturing device 115a being positioned inside the imaging channel 113a, in some embodiments, an image capturing device may be positioned outside the imaging channel 113a. For example, another image capturing device may be positioned along the central axis 110 of the eye 102. Additionally or alternatively, an image capturing device may be positioned within the imaging channel 113a such that the image capturing device has a corresponding optical imaging pathway normal to the eye (e.g., a direct line) such that no prism is needed for the optical imaging pathway.

In some embodiments, the optical illumination pathway 140 may include a path along which the illumination source 117a is configured to illuminate. Additionally or alternatively, the optical illumination pathway 140 may include an optical path that the illumination source 117a utilizes to illuminate target portions of the eye 102, such as the iridocorneal angle 145. In these or other embodiments, the illuminated portions of the eye 102 may correspond to imaged portions and/or help provide image data with topographical information.

In some embodiments, the optical imaging pathway 135 and the optical illumination pathway 140 may be directed towards the iridocorneal angle 145 at different angles relative to each other. For example, an approach angle for the optical imaging pathway 135 and an approach angle for the optical illumination pathway 140 may be different in one or more planes. In some embodiments, for example, the optical imaging pathway 135 and the optical illumination pathway 140 may approach the iridocorneal angle 145 at different angles in the sagittal plane as illustrated in FIG. 1B. Additionally or alternatively, the optical imaging pathway 135 and the optical illumination pathway 140 may approach the iridocorneal angle 145 at different angles in the coronal plane (e.g., as illustrated, for example, in FIG. 2A). In these or other embodiments, the optical imaging pathway 135 and the optical illumination pathway 140 may approach the iridocorneal angle 145 at different angles relative to each other such that topographical information may be obtained for any of the cornea 155, iris 150 and iridocorneal angle 145. The angular relationship between the optical imaging pathway 135 and the optical illumination pathway 140 with respect to topographical information is described in greater detail below.

In some embodiments, patterned illumination may be emitted by the illumination source 117a from within the imaging channel 113a along the optical illumination pathway 140, which may be non-coaxial to the optical imaging pathway 135 also within the imaging channel 113a. For example, the optical imaging pathway 135 may be positioned at a center portion of the optical imaging channels (e.g., along the center channel axis 107a), and the optical illumination pathway 140 may be positioned at an off-center portion of the imaging channel 113a, while in other embodiments vice-versa, or in other embodiments both optical pathways 135/140 positioned at different off-center portions within the imaging channel 113a. Additionally or alternatively, using a polarized beam splitter, the optical illumination pathway 140 may be further angled relative to the optical imaging pathway 135.

Additionally or alternatively, in some embodiments, the patterned illumination may be emitted from outside the imaging channel 113a. For example, the illumination source 117a may be positioned along or outside of a perimeter of the imaging channel 113a or at some other suitable position within or along an outside surface of the support structure 103 of FIG. 1A. In these or other embodiments, more extreme angles for the optical illumination pathway 140 may be achieved outside of the imaging channel 113a and may provide additional space within the imaging channel 113a.

Although some embodiments may include internal reference illumination beams, it is not required that the illumination be split into a reference beam and a beam for illuminating an area to be imaged, nor does the optical imaging device 100 depend on interferometry the way optical coherence tomography fundamentally depends on interferometry using a reference beam.

Modifications, additions, or omissions may be made to the embodiments of FIG. 1B without departing from the scope of the present disclosure. For example, in some embodiments, the imaging channel 113a (and any other imaging channel described in the present disclosure) may include any number of other components that may not be explicitly illustrated or described. Additionally or alternatively, the optical imaging pathway 135 and the optical illumination pathway 140 may approach an area of the eye 102 at other suitable angles than may be explicitly illustrated or described. Additionally or alternatively, a variety of different areas of the eye may be imaged to obtain topographic information, for example, the iris 150, the cornea 155, and other suitable areas of the eye.

FIG. 2A illustrates a front schematic view of the eye 102 of FIG. 1B, including an embodiment with multiple example optical pathways for imaging the iridocorneal angle 145 of the eye 102, all arranged according to one or more embodiments of the present disclosure. As illustrated, FIG. 2A includes an optical imaging pathway 235 and an optical illumination pathway 240 relative to the prism 130 of the optical imaging device 100 in FIG. 1B and relative to various features of the eye 102, including the iridocorneal angle 145, the iris 150, and a sclera 160.

In some embodiments, the optical imaging pathway 235 may be the same as or similar to the optical imaging pathway 135 of FIG. 1B. Additionally or alternatively, the optical illumination pathway 240 may be the same as or similar to the optical illumination pathway 140 of FIG. 1B. In these or other embodiments, the optical imaging pathway 235 and the optical illumination pathway 240 may approach the iridocorneal angle 145 at different angles relative to each other such that topographical information may be obtained for any of the cornea 155 (of FIG. 1B), the iris 150, and the iridocorneal angle 145. Were the optical imaging pathway 235 and the optical illumination pathway 240 directed towards a same portion of the eye 102 in a parallel manner, a resultant image may include little to no topographical data (e.g., as explicitly shown or obtainable via post-imaging analysis).

In comparison, via imaging along the optical imaging pathway 235 that is not collinear with the optical illumination pathway 240, topographical information may be obtained in a resultant image taken by an image capturing device. The topographical information may include contours, peaks, valleys, slopes, shadows, colorations, shading, reflections, optical losses, refractive indices, and any other suitable indicator of eye topology as indicated in or extracted from the resultant image taken by the image capturing device. For example, because the optical illumination pathway 240 is coming in at an angle relative to the optical imaging pathway 235, any variations in the topographical surface of the iridocorneal surface may be reflected in the shadows cast by the peaks, valleys, etc. in the topography of the iridocorneal surface.

The topographical information may be determined and/or further analyzed according to software analysis. For example, in some embodiments, the relative angles of the optical imaging pathway 235 and the optical illumination pathway 240 may be known variables that aid in topographic analysis of the eye 102. In these or other embodiments, a difference between the respective approach angles (e.g., at the iridocorneal angle 145) for the optical imaging pathway 235 and the optical illumination pathway 240 may be approximately 45 degrees, approximately 35 degrees, approximately 25 degrees, approximately 15 degrees, approximately 5 degrees, and any other suitable angular difference. In these or other embodiments, the optical illumination pathways 240a and 240b may correspond to a same imaging channel (e.g., share the same imaging channel). However, in other embodiments, the optical illumination pathways 240a and 240b may correspond to different imaging channels.

Modifications, additions, or omissions may be made to the embodiments of FIG. 2A without departing from the scope of the present disclosure. For example, more or fewer numbers of the optical imaging pathway 235 and the optical illumination pathway 240 may be utilized than may be explicitly illustrated or described. Additionally or alternatively, the optical imaging pathway 235 and the optical illumination pathway 240 may approach an area of the eye 102 at different angles than may be explicitly illustrated or described.

FIG. 2B illustrates a front schematic view of the eye 102 of FIG. 1B, including another embodiment with multiple example optical pathways for imaging the iridocorneal angle 145 of the eye 102, all arranged according to one or more embodiments of the present disclosure. As illustrated, FIG. 2B includes the optical imaging pathway 235 of FIG. 2A and optical illumination pathways 240a and 240b relative to the prism 130 of the optical imaging device 100 in FIG. 1B and relative to various features of the eye 102, including the iridocorneal angle 145, the iris 150, and a sclera 160.

In some embodiments, the optical illumination pathways 240a and 240b may be the same as or similar to the optical illumination pathway 240 of FIG. 2A. In these or other embodiments, the optical illumination pathways 240a and 240b may be directed towards the iridocorneal angle 145 at approximately mirrored angles relative to the optical imaging pathway 235. Thus, in some embodiments, the optical imaging pathway 235 may be directed towards an area of the eye 102 illuminated from multiple sides of the optical imaging pathway 235. In these or other embodiments, the optical imaging pathways 235a and 235b may correspond to a same imaging channel (e.g., share the same imaging channel). However, in some embodiments, the optical imaging pathways 235a and 235b may correspond to different imaging channels. Additionally or alternatively, in some embodiments, the optical illumination pathways 240a and 240b may be illuminated sequentially with images taken along the optical imaging pathway 235 for each of the sequential illuminations, such that the topographical data of the iridocorneal surface may be determined based on illumination coming from two different directions.

Modifications, additions, or omissions may be made to the embodiments of FIG. 2B without departing from the scope of the present disclosure. For example, more or fewer numbers of the optical imaging pathway 235 and the optical illumination pathways 240 may be utilized than may be explicitly illustrated or described. Additionally or alternatively, the optical imaging pathway 235 and the optical illumination pathway 240 may approach an area of the eye 102 at different angles than may be explicitly illustrated or described. Additionally or alternatively, additional prisms 130 may be utilized than may be explicitly illustrated or described.

FIG. 2C illustrates a front schematic view of the eye 102 of FIG. 1B, including yet another embodiment with multiple example optical pathways for imaging the iridocorneal angle 145 of the eye 102, all arranged according to one or more embodiments of the present disclosure. As illustrated, FIG. 2C includes optical imaging pathways 235a and 235b and optical illumination pathways 240a and 240b relative to prisms 130a-130c and relative to various features of the eye 102, including the iridocorneal angle 145, the iris 150, and the sclera 160.

The optical imaging pathways 235a and 235b may be the same as or similar to the optical imaging pathway 235 of FIGS. 2A-2B. In these or other embodiments, the optical imaging pathways 235a and 235b may correspond to a same imaging channel (e.g., share the same imaging channel). However, in some embodiments, the optical imaging pathways 235a and 235b may correspond to different imaging channels. Additionally or alternatively, prisms 130a-130c may be the same as or similar to the prism 130 of FIGS. 1A-1B and FIGS. 2A-2B. In these or other embodiments, the prisms 130a-130c may correspond to a same imaging channel (e.g., share the same imaging channel). However, in some embodiments, the prisms 130a-130c may correspond to different imaging channels. In these or other embodiments, multiple areas of the iridocorneal angle 145 around the eye 102 may be imaged, for example, at a first iridocorneal angle 145a and a second iridocorneal angle 145b.

In some embodiments, different optical pathways may correspond to different portions of the iridocorneal angle 145. As illustrated, for example, the optical imaging pathway 235a and the optical illumination pathway 240b may correspond to the first iridocorneal angle 145a. Additionally or alternatively, the optical imaging pathway 235b and the optical illumination pathway 240a may correspond to the second iridocorneal angle 145b.

In some embodiments, the optical imaging pathways 235a and 235b may impinge different prisms 130a and 130b, respectively. Additionally or alternatively, the optical illumination pathways 240a and 240b may both impinge the prism 130c. In these or other embodiments, any of the prisms 130a-130c may be positioned relative to each other. For example, as illustrated, the prism 130a is positioned proximate to the prism 130c; the prism 130b is positioned proximate to the prism 130c; and the prism 130c is positioned proximate to both the prism 130a and the prism 130b. However, other suitable arrangements are contemplated. For example, any of the prisms 130 may be positioned beyond a proximate distance to another prism 130. In these or other embodiments, the term “proximate” in reference to positional prism proximity may include a distance ranging from direct contact (zero mm) to a threshold distance of 5 mm, written as a closed range of [0,5] mm.

Modifications, additions, or omissions may be made to the embodiments of FIG. 2C without departing from the scope of the present disclosure. For example, more or fewer numbers of the prisms 130, the optical imaging pathways 235, and the optical illumination pathways 240 may be utilized than may be explicitly illustrated or described. Additionally or alternatively, the optical imaging pathways 235 and the optical illumination pathways 240 may be directed towards different areas of the iridocorneal angle 145 and/or impinge different prisms 130 than may be explicitly illustrated or described. For example, other suitable combinations of the prisms 130, the optical imaging pathways 235, and the optical illumination pathways 240 may be employed.

FIG. 2D illustrates a front schematic view of the eye 102 of FIG. 1B, including yet another embodiment with multiple example optical pathways for imaging the iridocorneal angle 145 of the eye 102, all arranged according to one or more embodiments of the present disclosure. As illustrated, FIG. 2D includes the optical imaging pathways 235a and 235b and optical illumination pathways 240a and 240b of FIG. 2C relative to the prisms 130a-130b and relative to various features of the eye 102 in FIG. 1B, including the iridocorneal angle 145, the iris 150, and the sclera 160. In these or other embodiments, any of the optical imaging pathways 235a and 235b, the optical illumination pathways 240a and 240b, and/or the prisms 130a-130b may correspond to a same imaging channel or different imaging channels.

In some embodiments, different optical pathways may correspond to different portions of the iridocorneal angle 145. As illustrated, for example, the optical imaging pathway 235a and the optical illumination pathway 240b may correspond to the first iridocorneal angle 145a. Additionally or alternatively, the optical imaging pathway 235b and the optical illumination pathway 240a may correspond to the second iridocorneal angle 145b. In some embodiments, the optical imaging pathways 235a and 235b may impinge different prisms 130a and 130b, respectively. Additionally or alternatively, the optical illumination pathways 240a and 240b may impinge different prisms 130a and 130b, respectively.

Modifications, additions, or omissions may be made to the embodiments of FIG. 2D without departing from the scope of the present disclosure. For example, more or fewer numbers of the prisms 130, the optical imaging pathways 235, and the optical illumination pathways 240 may be utilized than may be explicitly illustrated or described. Additionally or alternatively, the optical imaging pathways 235 and the optical illumination pathways 240 may be directed towards different areas of the iridocorneal angle 145 and/or impinge different prisms 130 than may be explicitly illustrated or described. For example, other suitable combinations of the prisms 130, the optical imaging pathways 235, and the optical illumination pathways 240 may be employed.

FIG. 2E illustrates a front schematic view of the eye 102 of FIG. 1B, including yet another embodiment with multiple example optical pathways for imaging the iridocorneal angle 145 of the eye 102, all arranged according to one or more embodiments of the present disclosure. As illustrated, FIG. 2E includes the optical imaging pathways 235a and 235b and optical illumination pathways 240a and 240b of FIGS. 2C-2D relative to the prisms 130a-130b and relative to various features of the eye 102 in FIG. 1B, including the iridocorneal angle 145, the iris 150, and the cornea 155. In these or other embodiments, any of the optical imaging pathways 235a and 235b, the optical illumination pathways 240a and 240b, and/or the prisms 130a-130b may correspond to a same imaging channel or different imaging channels.

In some embodiments, different optical pathways may correspond to a same portion of the iridocorneal angle 145. As illustrated, for example, each of the optical imaging pathways 235a and 235b and optical illumination pathways 240a and 240b may correspond to the iridocorneal angle 145 at a same area. Additionally or alternatively, the optical imaging pathways 235a and 235b may impinge different prisms 130a and 130b, respectively. Additionally or alternatively, the optical illumination pathways 240a and 240b may impinge different prisms 130a and 130b, respectively.

In some embodiments, the optical illumination pathways 240a and 240b may be illuminated sequentially with images taken along one or both of the optical imaging pathways 235a and/or 235b for each of the sequential illuminations, such that the topographical data of the iridocorneal surface may be determined based on illumination coming from the two different directions.

Modifications, additions, or omissions may be made to the embodiments of FIG. 2E without departing from the scope of the present disclosure. For example, more or fewer numbers of the prisms 130, the optical imaging pathways 235, and the optical illumination pathways 240 may be utilized than may be explicitly illustrated or described. Additionally or alternatively, the optical imaging pathways 235 and the optical illumination pathways 240 may be directed towards different areas of the iridocorneal angle 145 and/or impinge different prisms 130 than may be explicitly illustrated or described. For example, other suitable combinations of the prisms 130, the optical imaging pathways 235, and the optical illumination pathways 240 may be employed.

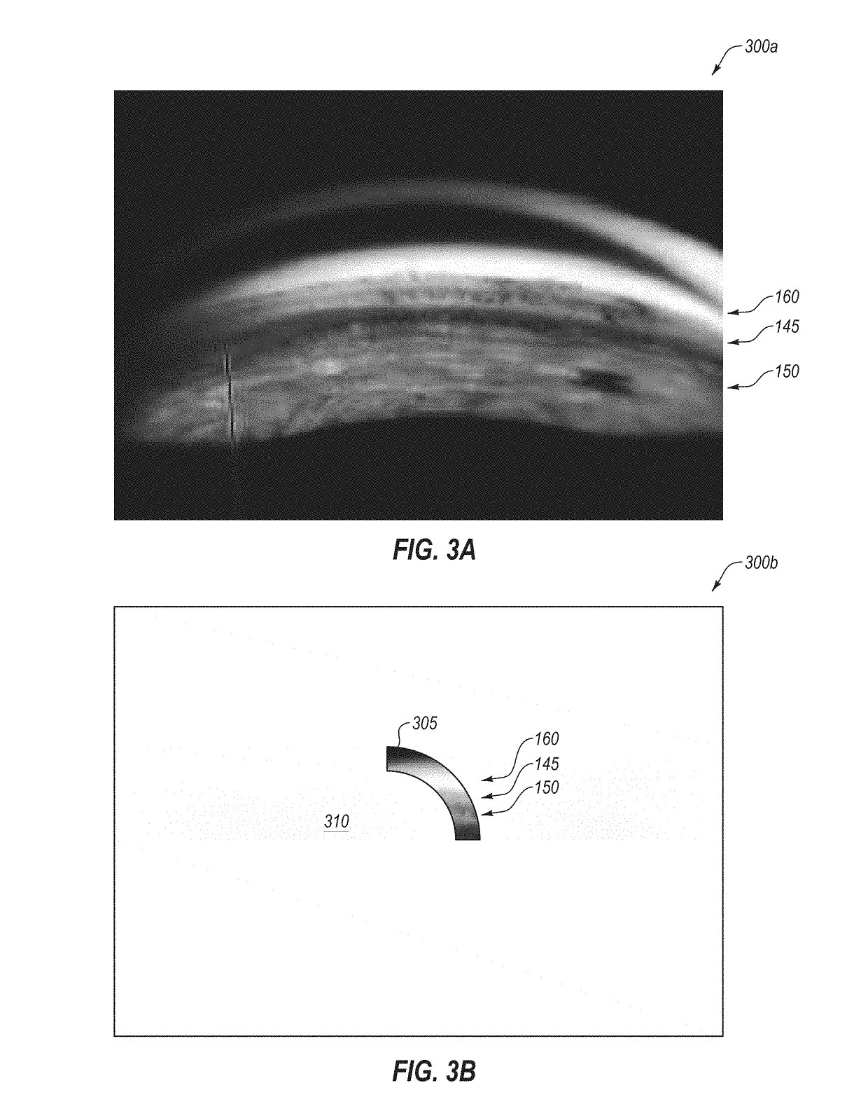

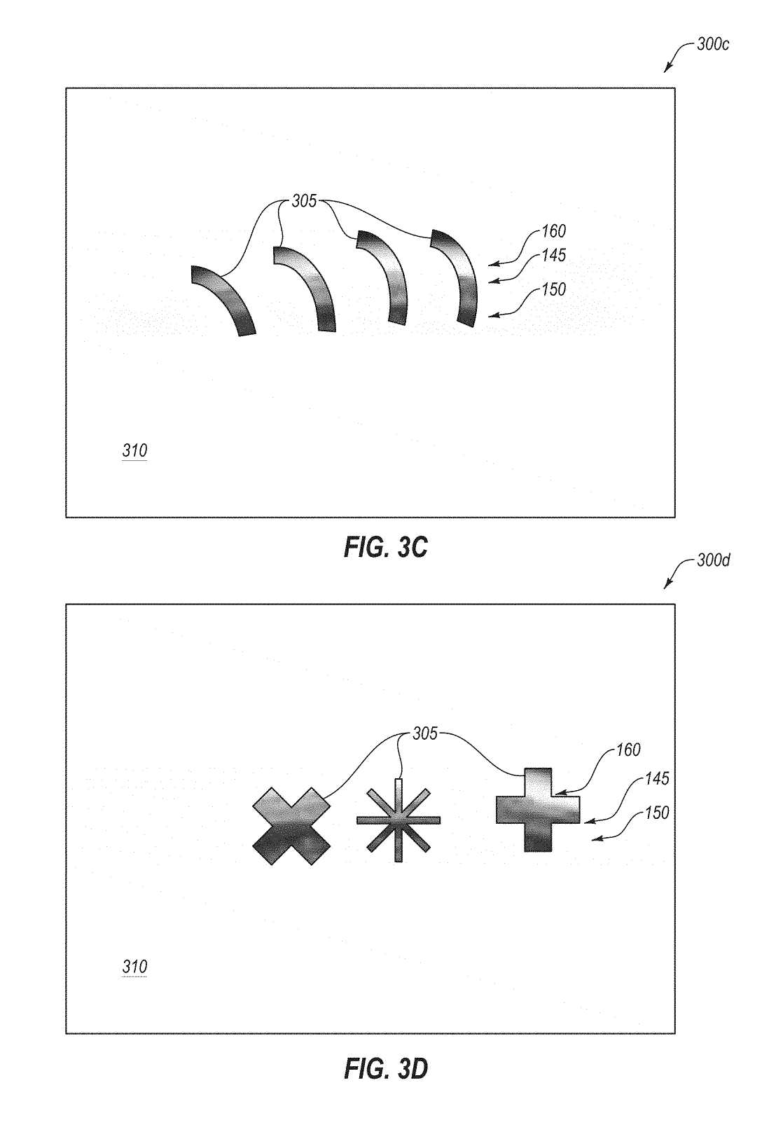

FIG. 3A illustrates example image data 300a of a portion of the iridocorneal angle 145 obtained using diffuse illumination, for example employing the configuration of FIG. 2A. As illustrated in the image data 300a, various tissue layers are depicted between a sclera 160 and the iris 150, including the iridocorneal angle 145. However, little to no topographical information may be obtained with diffuse illumination.

FIG. 3B illustrates example image data 300b of the portion of the iridocorneal angle 145 in FIG. 3A obtained using patterned illumination, all arranged according to one or more embodiments of the present disclosure. As illustrated, the image data 300b may include an imaged portion 305 and an un-imaged portion 310. The imaged portion 305 may include one or more of the tissue layers of FIG. 3A including the sclera 160, the iridocorneal angle 145, and the iris 150.

In some embodiments, the imaged portion 305 may be sized and shaped based on an illumination pattern, e.g., as generated by an illumination source. Additionally or alternatively, the imaged portion 305 may be sized and shaped based on the curvature of the portion of the eye to be imaged. Additionally or alternatively, the imaged portion 305 may be sized and shaped based on an angle of the optical illumination pathway relative to the optical imaging pathway. For example, as illustrated, the imaged portion 305 includes a curved slit shape due to both the slit pattern and the non-collinear nature of the optical illumination pathway relative to the optical imaging pathway. Were the optical imaging pathway collinear with the optical illumination pathway, the slit would be a vertical or straight slit according to the slit illumination pattern.

In some embodiments, the illumination pattern may be known, thereby helping the imaging device to digitally detect topographical features of the iridocorneal angle 145. Additionally or alternatively, the illumination pattern may correspond to fixed spatial coordinates and other known parameters such that upon detection of the illumination pattern against the topographical features of the iridocorneal angle 145, various details and intricacies of tissue formations, contours, layers, and configurations (geometric, spatial, or otherwise) corresponding to the iridocorneal angle 145 may be detected or calculated via software analysis.

In some embodiments, the illumination pattern may include a slit beam of white light, while in other embodiments the illumination pattern may include a shadow slit (e.g., negative illumination). In some embodiments, the relative angle and/or position of the illumination pattern (of the optical illumination pathway) relative to the optical imaging pathway may be fixed or known, thus facilitating automated software-based analysis of topographical features. In other embodiments, the illumination pattern may include narrow-band or multi-spectral pattern illumination, either simultaneous with or not simultaneous with broad-band or white light illumination of a larger section of the iridocorneal angle 145. In some embodiments, the illumination pattern may be generated by illuminating a narrow area of the iridocorneal angle 145 with one or more wavelengths of light more brightly than the surrounding tissue is illuminated. In other embodiments, the illumination pattern is generated by blocking or masking illumination of the iridocorneal angle 145 in order to reduce illumination of one narrow section of the iridocorneal angle 145 in a predefined manner relative to the illumination of the surrounding tissue.

For example, the illumination pattern may be flashed once diffusely, and then again upon masking, for example, with a slit. In these or other embodiments, a shadow may exist at an edge of the main illumination (e.g., the imaged portion 305). Thus, in some embodiments, the un-imaged portion 310 may appear as a shadow and encompass the imaged portion 305. However, with overlapping images in rapid sequence, the shadows or the un-imaged portion 310 may be placed in overlapping regions such that software may detect the shadow and subtract it out when stitching all the images together for the composite image. In some embodiments, non-visible light may be used in all or some of the illumination patterns. Additionally or alternatively, non-visible light, such as infrared, may be used in substitute of other illumination types or simultaneously in combination with other illumination types, such as white light.

Modifications, additions, or omissions may be made to the embodiments of FIG. 3B without departing from the scope of the present disclosure. For example, more imaged portions 305 may be utilized than may be explicitly illustrated or described. Additionally or alternatively, the imaged portion 305 may be sized and shaped according to a different illumination pattern than may be explicitly illustrated or described.

FIG. 3C illustrates example image data 300c of the portion of the iridocorneal angle 145 in FIG. 3A obtained using multiple-patterned illumination, all arranged according to one or more embodiments of the present disclosure. In some embodiments, the multiple imaged portions 305 may correspond to multiple patterns of illumination and/or multiple illumination sources. In these or other embodiments, multiple imaged portions 305 may aid in creating composite images and/or obtaining topographical information at multiple points. For example, in some embodiments, the iridocorneal angle 145 may be imaged with multiple partially overlapping images in order to create a continuous or representative composite image of the circumferential iridocorneal angle 145 via an en face view. Additionally or alternatively, patterned illumination may be applied in a region of imaging overlap such that the pattern illumination may create artifacts in the en face image of the overlap region when imaged from one imaging channel, but that same region of the iridocorneal angle 145 may be imaged by a different imaging channel without concurrent pattern illumination of that same region. By observing the contours of the pattern, the topography of the iridocorneal angle 145 may be obtained in a similar manner as described above with respect to the shadows associated with a beam of illumination.

Modifications, additions, or omissions may be made to the embodiments of FIG. 3C without departing from the scope of the present disclosure. For example, more or fewer imaged portions 305 may be utilized than may be explicitly illustrated or described. Additionally or alternatively, any of the imaged portions 305 may be sized and shaped according to a different illumination pattern than may be explicitly illustrated or described.

FIG. 3D illustrates example image data 300d of the portion of the iridocorneal angle 145 in FIG. 3A obtained using differing patterns in multiple-patterned illumination, all arranged according to one or more embodiments of the present disclosure. In some embodiments, multiple distinct patterns and/or wavelengths of illumination may be used to illuminate a given section of the iridocorneal angle 145 sequentially in order to gain more topographical information than could be revealed with a single illumination pattern. For example, different wavelengths of light may penetrate iridocorneal angle tissue to different degrees, providing information about different layers of tissue, and each wavelength may utilize a distinct illumination pattern. In some embodiments, the different wavelengths may be visible or non-visible light, for example infrared.

In some embodiments, a given section of the iridocorneal angle 145 may be illuminated by patterns of illumination from two or more different directions, such that two or more optical illumination pathways are non-coaxial with each other and also non-coaxial with an optical imaging pathway. Additionally or alternatively, the illumination patterns need not be the same among all the illumination patterns. For example, one illumination pattern may include a polka-dot pattern, while another may be a slit-beam pattern, and another a striped pattern, an “X” pattern, an asterisk (*) pattern, a star pattern, a plus (+) symbol pattern, a minus (−) symbol pattern, a “T” pattern, and/or any other suitable pattern, including between positive and negative illumination. In these or other embodiments, the illumination pattern may move due to either movement of the illumination source as a whole or due to actuated movement of components within the illumination source. For example, a slit or multiple slits may move to create a different illumination pattern or to create a different angle of the illumination pattern. The movement may be done once or multiple times, while in other embodiments continuously, to create a scanning motion of the illumination pattern.

In some embodiments, multiple patterned illumination may be applied to multiple separate sections of the iridocorneal angle in order to obtain representative measurements of the iridocorneal angle topography without measuring topographical information three hundred and sixty degrees around the angle. For example, four or six or eight evenly spaced sections of the iridocorneal angle may be analyzed with pattern illumination in order to provide a representative analysis of the topography. In these or other embodiments, a full three hundred and sixty degree view may or may not be achieved, nor may it be necessary for certain purposes. Additionally or alternatively, in some embodiments, the images may or may not be overlapping.

In some embodiments, topographical data may be derived from direct measurement of the illumination tissue contour, while in other embodiments, topographical data may be derived from objective measurement of light scatter by illuminated tissue with known angles or positions of illumination patterns, e.g., relative to the optical imaging pathway 135 of FIG. 1B. Additionally or alternatively, in some embodiments, topographical data may be derived based on the reflectance, scattering, and/or absorption of certain wavelengths of illumination at various depths of tissue.

Modifications, additions, or omissions may be made to the embodiments of FIG. 3D without departing from the scope of the present disclosure. For example, more or fewer imaged portions 305 may be utilized than may be explicitly illustrated or described. Additionally or alternatively, any of the imaged portions 305 may be sized and shaped according to a different illumination pattern than may be explicitly illustrated or described.

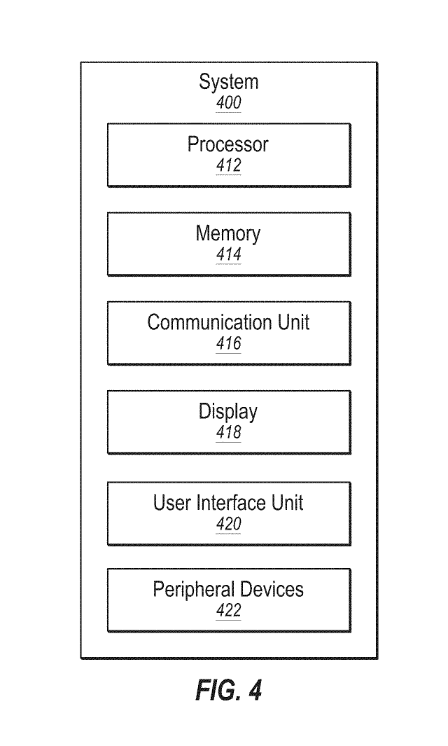

FIG. 4 illustrates an example system 400 that may be used in patterned beam analysis of the eye. The system 400 may be arranged in accordance with at least one embodiment described in the present disclosure. The system 400 may include a processor 412, memory 414, a communication unit 416, a display 418, a user interface unit 420, and a peripheral device 422, which all may be communicatively coupled. In some embodiments, the system 400 may be part of any of the systems or devices described in this disclosure.

Generally, the processor 412 may include any suitable special-purpose or general-purpose computer, computing entity, or processing device including various computer hardware or software modules and may be configured to execute instructions stored on any applicable computer-readable storage media. For example, the processor 412 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data.

Although illustrated as a single processor in FIG. 4, it is understood that the processor 412 may include any number of processors distributed across any number of networks or physical locations that are configured to perform individually or collectively any number of operations described in this disclosure. In some embodiments, the processor 412 may interpret and/or execute program instructions and/or process data stored in the memory 414. In some embodiments, the processor 412 may execute the program instructions stored in the memory 414.

For example, in some embodiments, the processor 412 may execute program instructions stored in the memory 414 that are related to determining whether generated sensory data indicates an event and/or determining whether the event is sufficient to determine that the user is viewing a display of a device such that the system 400 may perform or direct the performance of the operations associated therewith as directed by the instructions. In these and other embodiments, instructions may be used to perform one or more operations or functions described in the present disclosure.

The memory 414 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 412. By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 412 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.3d 1346 (Fed. Cir. 2007). Combinations of the above may also be included within the scope of computer-readable media.

The communication unit 416 may include any component, device, system, or combination thereof that is configured to transmit or receive information over a network. In some embodiments, the communication unit 416 may communicate with other devices at other locations, the same location, or even other components within the same system. For example, the communication unit 416 may include a modem, a network card (wireless or wired), an infrared communication device, a wireless communication device (such as an antenna), and/or chipset (such as a Bluetooth device, an 802.6 device (e.g., Metropolitan Area Network (MAN)), a Wi-Fi device, a WiMax device, cellular communication facilities, etc.), and/or the like. The communication unit 416 may permit data to be exchanged with a network and/or any other devices or systems described in the present disclosure.

The display 418 may be configured as one or more displays, like an LCD, LED, or other type of display. For example, the display 418 may be configured to present measurements, indicate warning notices, show tolerance ranges, display whether good/bad eye tissues are determined, and other data as directed by the processor 412.

The user interface unit 420 may include any device to allow a user to interface with the system 400. For example, the user interface unit 420 may include a mouse, a track pad, a keyboard, buttons, and/or a touchscreen, among other devices. The user interface unit 420 may receive input from a user and provide the input to the processor 412. In some embodiments, the user interface unit 420 and the display 418 may be combined.

The peripheral devices 422 may include one or more devices. For example, the peripheral devices may include a sensor, a microphone, and/or a speaker, among other peripheral devices. As examples, the sensor may be configured to sense changes in light, sound, motion, rotation, position, orientation, magnetization, acceleration, tilt, vibration, etc., e.g., as relating to an eye of a patient. Additionally or alternatively, the sensor may be part of or communicatively coupled to the optical imaging device as described in the present disclosure.

Modifications, additions, or omissions may be made to the system 400 without departing from the scope of the present disclosure. For example, in some embodiments, the system 400 may include any number of other components that may not be explicitly illustrated or described. Further, depending on certain implementations, the system 400 may not include one or more of the components illustrated and described.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner. Additionally, the terms “about,” “substantially,” or “approximately” should be interpreted to mean a value within 10% of an actual value, for example, values like 3 mm or 100% (percent).

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. An optical imaging device, comprising:
a support structure;
a plurality of imaging channels, each imaging channel of the plurality of imaging channels including a discrete optical imaging pathway, the plurality of imaging channels disposed within the support structure, and the plurality of imaging channels aimed at different angles relative to each other;
a plurality of illumination sources corresponding respectively to the plurality of imaging channels, each illumination source of the plurality of illumination sources configured to emit an illumination pattern along a discrete optical illumination pathway positioned non-coaxially relative to the discrete optical imaging pathway of each imaging channel of the plurality of imaging channels; and
a plurality of image capturing devices, each image capturing device of the plurality of image capturing devices respectively associated with one of the plurality of imaging channels to capture digital photograph images of respective portions of an iridocorneal angle of an eye to generate a topographical profile of the iridocorneal angle revealed by the plurality of illumination sources.

2. The optical imaging device of claim 1, wherein the digital photograph images include topographical information of the iridocorneal angle at multiple positions around the eye.

3. The optical imaging device of claim 1, wherein the digital photograph images overlap each other and are stored in a storage device of the optical imaging device for stitching together such that the digital photograph images form a composite image of up to a 360 degree view of the iridocorneal angle.

4. The optical imaging device of claim 1, further comprising one or more prisms disposed within at least one imaging channel of the plurality of imaging channels.

5. The optical imaging device of claim 4, wherein the one or more prisms includes a first prism and a second prism positioned proximate to each other within the at least one imaging channel.

6. The optical imaging device of claim 4, wherein both the discrete optical imaging pathway and the discrete optical illumination pathway correspond to the at least one imaging channel and impinge the one or more prisms of the at least one imaging channel such that both the discrete optical imaging pathway and the discrete optical illumination pathway are directed, at different angles relative to each other, towards the iridocorneal angle of the eye.

7. The optical imaging device of claim 6, wherein:
the one or more prisms includes a first prism configured to direct the discrete optical imaging pathway towards the iridocorneal angle of the eye at a first location; and
the one or more prisms includes a second prism configured to direct the discrete optical illumination pathway towards the iridocorneal angle of the eye at the first location.

8. The optical imaging device of claim 6, wherein the discrete optical illumination pathway of the at least one imaging channel of the plurality of imaging channels is a first optical illumination pathway corresponding to a first illumination source within the at least one imaging channel, and the optical imaging device further comprises:
a second optical illumination pathway of the at least one imaging channel, the second optical illumination pathway corresponding to a second illumination source within the at least one imaging channel, and the second optical illumination pathway impinging the one or more prisms of the at least one imaging channel such that the second optical illumination pathway is directed towards the iridocorneal angle of the eye at a different angle than both the first optical illumination pathway and the discrete optical imaging pathway.

9. The optical imaging device of claim 8, wherein:
the first illumination source and the second illumination source sequentially emit illumination along the first optical illumination pathway and the second optical illumination pathway, respectively; and
an image capturing device of the plurality of image capturing devices captures at least one digital photograph image of the iridocorneal angle revealed by the illumination sequentially emitted from the first illumination source and the second illumination source.

10. The optical imaging device of claim 8, wherein the discrete optical imaging pathway of the at least one imaging channel of the plurality of imaging channels is a first optical imaging pathway, and the optical imaging device further comprises:
a second optical imaging pathway of the at least one imaging channel, the second optical imaging pathway impinging the one or more prisms of the at least one imaging channel such that the second optical illumination pathway is directed towards the iridocorneal angle of the eye at a different angle than both the first optical illumination pathway and the discrete optical imaging pathway.

11. The optical imaging device of claim 10, wherein the first optical imaging pathway and the second optical imaging pathway are oriented towards different areas of the iridocorneal angle so as to enable one or more image capturing devices of the plurality of image capturing devices to image the different areas of the iridocorneal angle via the first optical imaging pathway and the second optical imaging pathway.

12. The optical imaging device of claim 10, wherein the first optical illumination pathway is oriented to illuminate an area of the iridocorneal angle that corresponds to one or more of the second optical illumination pathway, the first optical imaging pathway, and the second optical imaging pathway.

13. The optical imaging device of claim 10, wherein the first optical illumination pathway impinges a same prism of the one or more prisms as impinged by one or more of the second optical illumination pathway, the first optical imaging pathway, and the second optical imaging pathway.

14. The optical imaging device of claim 1, wherein at least one image capturing device of the plurality of image capturing devices is a common imaging sensor for two or more discrete optical imaging pathways.

15. The optical imaging device of claim 1, wherein at least one illumination source of the plurality of illumination sources emits positive illumination.

16. The optical imaging device of claim 1, wherein at least one illumination source of the plurality of illumination sources emits a slit-patterned illumination.

17. The optical imaging device of claim 1, wherein:
the plurality of illumination sources emit patterned illumination; and
when at least one pattern of illumination of the plurality of illumination sources is projected onto the eye at an imaged portion for a first image, an un-imaged portion outside the at least one pattern of illumination is positioned within an overlap region.

18. The optical imaging device of claim 17, wherein:
the overlap region is configured to be subsequently imaged in a second image; and
the first image and the second image are stored in a storage device of the optical imaging device for stitching together such that the first image and the second image form a composite image.

19. A system comprising:
one or more processors configured to receive optical imaging data; and
an optical imaging device configured to generate optical imaging data, the optical imaging device communicatively coupled to the one or more processors, and the optical imaging device comprising:
a support structure;
a plurality of imaging channels, each imaging channel of the plurality of imaging channels including a discrete optical imaging pathway, the plurality of imaging channels disposed within the support structure, and the plurality of imaging channels aimed at different angles relative to each other;
a plurality of illumination sources corresponding respectively to the plurality of imaging channels, each illumination source of the plurality of illumination sources configured to emit an illumination pattern along a discrete optical illumination pathway positioned non-coaxially relative to the discrete optical imaging pathway of each imaging channel of the plurality of imaging channels; and
a plurality of image capturing devices, each image capturing device of the plurality of image capturing devices respectively associated with one of the plurality of imaging channels to capture digital photograph images of respective portions of an iridocorneal angle of an eye to generate a topographical profile of the iridocorneal angle revealed by the plurality of illumination sources.

20. The system of claim 18, wherein the digital photograph images include topographical information of the iridocorneal angle at multiple positions around the eye.
